SEQUENCE LISTING (2 sequences)

SEQ ID NO: 1 - protein, 10 aa, Artificial Sequence
  Chemically Synthesized
WYLNSAGYLL

SEQ ID NO: 2 - protein, 9 aa, Artificial Sequence
  Chemically Synthesized
ASQKRPSQR

(19) United States
(12) Patent Application Publication
(10) Pub. No.: US 2010/0184638 A1
(43) Pub. Date: Jul. 22, 2010

(54) Galanin Receptors and Brain Injury

(75) Inventor: David WYNICK, Bristol (GB)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: NeuroTargets, Ltd., Guildford (GB)

(21) Appl. No.: 12/606,439

(22) Filed: Oct. 27, 2009

Related U.S. Application Data
(62) Division of application No. 10/589,533, filed on May 15, 2007, now abandoned.

(30) Foreign Application Priority Data
Feb. 17, 2004 (GB) 0403509.3

(51) Int. Cl. A61K 38/00; C12N 5/00; G01N 33/53

(52) U.S. Cl. 514/2; 435/375; 435/7.2

(57) ABSTRACT

There is provided the use of a GALR2-specific agonist in the preparation of a medicament for the prevention or treatment of brain injury, damage or disease, wherein the brain injury or damage is caused by one of: embolic, thrombotic or haem-orrhagic stroke; direct or indirect trauma or surgery to the brain or spinal cord; ischaemic or embolic damage to the brain during cardiopulmonary bypass surgery or renai dialysis; reperfusion brain damage following myocardial infarction; brain disease; chemical damage as the result of excess alcohol consumption or administration of chemotherapy agents for cancer treatment; radiation damage; or immunological damage as the result of bacterial or virai infection. The brain disease may be one of Alzheimer's Disease, Parkinson's Disease, Multiple Sclerosis or variant Creutzfeld Jacob Disease.

This application is a divisional of U.S. patent application Ser. No. 10/589,533, filed Aug. 16, 2006, which is a national stage filing of PCT/GB2005/000188, filed Jan. 18, 2005, which claims priority to GB 0403509.3, filed Feb. 17, 2004, the disclosure of each of which is hereby incorporated by reference.

### TECHNICAL FIELD

This invention relates to the field of protecting the central nervous system from injury, damage or disease.

The invention relates especially, but not exclusively, to protecting or treating the brain from the deleterious effects of (a) embolic, thrombotic or haemorrhagic stroke; (b) direct or indirect trauma to the brain or spinal cord; (c) surgery to the brain or spinal cord; (d) ischaemic or embolic damage to the brain resulting from cardiopulmonary bypass surgery, renal dialysis and reperfusion brain damage following myocardial infarction; (e) diseases of the brain that involve neuronal damage and/or cell death, such as Alzheimer's Disease, Parkinson's Disease, Multiple Sclerosis, vCJD (variant Creutzfeld Jacob Disease); (1) immunological, chemical or radiation damage to the brain such as that caused by bacterial or viral infections, alcohol, chemotherapy for tumours and radiotherapy for tumours.

In particular, the invention relates to the use of ligands of the second galanin receptor subtype (GALR2), in the prevention or treatment of brain injury, damage or disease. Advantageously, a GALR2-specific agonist can be used to protect or treat a range of diseases of the central nervous system and would minimize or obviate potential side effects attributable to activation of GALR1 and/or GALR3. The invention also relates to drug discovery methods for determining candidate drugs for use in the prevention or treatment of brain injury, damage or disease, and to pharmaceutical compositions for the prevention or treatment of brain injury, damage or disease.

### BACKGROUND ART

### Stroke

Stroke is defined as a cardiovascular accident, including an embolic, thrombotic or haemorrhagic episode that causes an area of brain anoxia, leading to permanent brain damage with associated functional neurological impairment. There are no satisfactory treatments for the neurological effects, despite stroke being the third-largest cause of death in the Western world. Stroke is responsible for much of the physical disability observed in the elderly population and up to 30% of stroke patients require long-term assistance with daily activities. The number of strokes occurring annually in the US has been estimated at over 700,000 and in the UK, at any one time, 500,000 people have had a stroke at some time in their life. A number of neuroprotective agents have been developed to attempt to minimise the effects of a stroke but these have so far been disappointing in practice and are not in widespread or regular clinical use. These include, but are not limited to, the calcium channel antagonists nilvadipine (Nivadil®) from Fujisawa and nimodipine (Nimotop®) from Bayer; the antioxidants tirilazad (Freedox®) from Pharmacia & Upjohn and citicoline (CerAxon®) from Interneuron; and the protein kinase inhibitor fasudil (Eril™) from Asahi. In addition to calcium channel antagonists and free-radical scavengers, neuroprotective agents in development include N-methyl-D-aspartate (NMDA) antagonists, α-amino-3-hydroxy-5-methyl-4-isoxazolepropionate (AMPA) antagonists and other compounds designed to inhibit release of toxic neurotransmitters such as glutamate and glycine agonists.

### Forms of Traumatic or Surgical Brain Injury

A range of conditions exist, other than stroke, in which brain damage occurs. These include direct or indirect trauma or surgery to the brain or spinal cord, surgery involving cardiopulmonary bypass, renal dialysis and reperfusion following myocardial infarction. The most common of these occurs during or after coronary artery bypass graft (CABG). 600,000 CABG surgeries are performed each year in the USA and 25% of all cardiopulmonary bypass patients exhibit neurological deficits within 3 months after surgery.

Diseases that Damage the Brain

Alzheimer's disease (AD) is a huge health burden in the Western world. AD is the commonest form of dementia in the elderly and there are currently an estimated 20 million people worldwide who have the disease. The incidence of AD is expected to double over the next 25 years as the population of elderly people increases. The annual cost of caring for AD sufferers in the UK is in excess of £5.5 billion. To date, no known cure exists for the disease and few treatments (other than the acetylcoline esterase inhibitors) have been shown to substantially slow the progression of the disease.

Multiple Sclerosis (MS) is the most common disabling neurological disease among young adults and affects around 85,000 people in the UK and over half a million people in the Western World at any one time. MS is most often diagnosed in people between the ages of 20 and 40, and women are almost twice as likely to develop it as men. The disease seems to preferentially target people of Northern European descent. MS is an autoimmune disease characterized by loss of the myelin sheath surrounding neurons resulting in progressive neuronal dysfunction and neuronal cell loss. Patients experience a range of problems that may include visual disturbance and blindness, loss of motor and/or sensory function and problems with bowel and urinary function.

Other diseases known to cause neuronal damage and/or cell death include Parkinson's Disease and variant Creutzfeld Jacob Disease.

Other forms of brain injury include immunological, chemical or radiation damage such as that caused by bacterial or viral infections, alcohol, chemotherapy for tumours and radiotherapy for tumours.

### Galanin

The twenty-nine amino-acid neuropeptide galanin (Tatemoto et al. (1983) FEES Lett. 164 124-128) is widely expressed in both the central and peripheral nervous system and has strong inhibitory actions on synaptic transmission by reducing the release of a number of classical neurotransmitters (Fisone et al. (1987) Proc. Natl. Acad. Sci. USA 84 7339-7343; Misane et al. (1998) Eur. J. Neurosci. 10 1230-1240; Pieribone et al. (1995) Neurosci. 64 861-876; Hokfelt et al. (1998) Ann. N.Y. Acad. Sci. 863 252-263; Kinney et al. (1998) J. Neurosci. 18 3489-3500; Zini et al. (1993) Eur. J. Pharmacol. 245 1-7). These inhibitory actions result in a diverse range of physiological effects, including:

a) an impairment of working memory (Mastropaolo et al. (1988) Proc. Natl. Acad. Sci. USA 85 9841-9845) and long term potentiation (LTP, thought to be the electrophysiological correlate of memory) (Sakurai et al. (1996) Neurosci. Lett. 212 21-24);
b) a reduction in hippocampal excitability with a decreased predisposition to seizure activity (Mazarati et al. (1992) Brain Res. 589 164-166); and
c) a marked inhibition of nociceptive responses in the intact animal and after nerve injury (Wiesenfeld et al. (1992) Proc. Natl. Acad. Sci. USA 89 3 334-3337).

These neuromodulatory actions of galanin have long been regarded as the principal role played by the peptide in the nervous system. However, there is now a large body of evidence to indicate that injury to many of these neuronal systems markedly induces the expression of galanin at both the mRNA and peptide levels. Examples of such lesion studies include the up-regulation of galanin in:

a) the dorsal root ganglion (DRG) following peripheral nerve axotomy (Hokfelt et al. (1987) Neurosci. Lett. 83 217-220),
b) magnocellular secretory neurons of the hypothalamus after hypophysectomy (Villar et al. (1990) Neurosci. 36 181-199),
c) the dorsal raphe and thalamus after removal of the frontoparietal cortex (decortication) (Cortes et al. (1990) Proc. Natl. Acad. Sci. USA 87 7742-7746),
d) the molecular layer of the hippocampus after an entorhinal cortex lesion (Harrison & Henderson (1999) Neurosci. Lett. 266 41-44), and
e) the medial septum (MS) and vertical limb diagonal-band (vdB) after a fimbria formix bundle transection (Brecht et al. (1997) Brain Res. Mol. Brain Res. 48 7-16).

These studies have led a number of investigators to speculate that galanin might play a cell survival or growth promoting role in addition to its classical neuromodulatory effects.

To test this hypothesis, transgenic animals were generated, bearing loss- or gain-of-function mutations in the galanin gene (Bacon et al. (2002) Neuroreport 13 2129-2132; Holmes et al. (2000) Proc. Natl. Acad. Sci. USA 97 11563-11568; Steiner et al. (2001) Proc. Natl. Acad. Sci. USA 98 4184-4189; Blakeman et al. (2001) Neuroreport 12 423-425). Phenotypic analysis of galanin knockout animals demonstrated that, surprisingly, the peptide acts as a survival factor to subsets of neurons in the developing peripheral and central nervous system (Holmes, 2000; O′Meara et al. (2000) Proc. Natl. Acad. Sci. USA 97 11569-11574). Most recently, it has been demonstrated that this neuronal survival role is also relevant to the adult DRG. Sensory neurons are dependent upon galanin for neurite extension after injury, mediated by activation of the second galanin receptor subtype in a PKC-dependent manner (Mahoney et al. (2003) J. Neurosci. 23 416-421). It was therefore hypothesised that galanin might also act in a similar manner in the central nervous system, reducing cell death in animal models of brain injury, damage or disease.

WO92/12997 discloses the sequence of human galanin. There is a discussion of studies by other workers involving the administration of rat galanin or its N-terminal fragments to augment the effect of morphine. This patent application suggests that galanin can be expected to exhibit analgesic effects such that it may be administered alone or in combination with other analgesics. The application claims the use of galanin or its analogues in the treatment of pain and the use of galanin antagonists in the treatment of certain other conditions.

WO92/20709 discloses a number of putative galanin antagonists. The antagonists which are described are all based on the first 12 amino acids of galanin followed by partial sequences of other peptides i.e. chimeric peptides. Some may be agonists, some antagonists and some may be both depending on the receptor subtype. The application discloses that the antagonists may be useful for treatment of insulin-, growth hormone-, acetyl choline-, dopamine-, Substance P-, Somatostatin-, and noradrenaline-related conditions including Alzheimer's type dementia and intestinal disease, along with conditions in the fields of endocrinology, food intake, neurology and psychiatry. Such antagonists may also be useful as analgesics. The application discloses the results of studies using some of the antagonists described therein on various effects such as galanin inhibition of glucose stimulated insulin release; galanin induced inhibition of scopolamine induced acetylcholine (ACh) hippocampal release; galanin induced facilitation of the flexor reflex; the displacement of bound iodinated galantin in membrane binding studies. There is a suggestion in the application that the antagonists may be indicated for analgesia but there is no disclosure in the application of results to this effect. No positive or beneficial claims are made concerning the use of galanin agonists.

Ukai et al. (1995) Peptides 16 1283-1286 describes an investigation into the effects of galanin on memory processes in mice. The results suggest that galanin impairs memory and other cognitive functions and that intermediate doses of galanin specifically elicit amnesia. No positive or beneficial claims are made concerning the use of galanin agonists. JP-A-6172387 discloses a synthetic peptide and derivatives for effectively inhibiting the insulin-secretion suppressing action of galanin, expected to be useful as a galanin-antagonistic substance for the prevention and treatment of Alzheimer's Disease.

Bartfai et al. (1992) TiPS 13 312-317 is a review article summarising the knowledge of the actions of galanin at that time and describing a series of high-affinity galanin antagonists. The review indicates that galanin antagonists may be useful in the treatment of Alzheimer's Disease.

Wynick et al. (1993) Nature 364 529-532 discusses the involvement of galanin in basal and oestrogen-stimulated lactotroph function and the release of the hormone prolactin.

WO92/15681 discloses a peptide having the amino acid sequence of human galanin and DNA clones encoding the peptide. The application suggests that galanin may play a role in pancreatic activity and claims methods of modulating pancreatic activity, or of stimulating the production of growth hormone, the methods involving the use of the disclosed peptides.

WO92/15015 discloses DNA encoding human galanin and methods for the identification of galanin antagonists.

WO97/26853, US2003/0129702, US2003/0215823 and U.S. Pat. No. 6,586,191 disclose the isolation of the GALR2 (second galanin receptor subtype) cDNA encoding GALR2 and methods of identifying a chemical compound which specifically binds to GALR2. There is mention that GALR2 antagonists may be effective in the treatment of Alzheimer's Disease. There is no disclosure of methods of selecting a brain injury prevention or treatment compound, on the basis of whether or not a compound is a GALR2 agonist.

Crawley (1996) Life Sci. 58 2185-2199 is a review article summarising the knowledge of the actions of galanin at that time. It indicates that centrally administered galanin produces deficits in learning and memory tasks in rats and that the use of galanin antagonists may be useful in the treatment of Alzheimer's Disease. No mention was made of the use of a galanin agonist for treatment of Alzheimer's Disease.

Liu et al. (1994) J. Neurotrauma 11 73-82 describes the effect of intraventricular injection of galanin on the extent of traumatic brain injury (TBI) caused by central fluid percussion in rats and showed that galanin-treated rats had significantly less deficits in various sensory motor tasks. The paper attributes these effects to the neuromodulatory action of galanin, decreasing the release of excitatory amino acids such as glutamate. However, there was no difference in a memory test (Morris water maze test) between galanin-treated and -untreated rats.

Luo et al. (1995) Neuropeptide 28 161-166 is a study to examine the effects of acute section of the sciatic nerve on the excitability of the flexor reflex in decerebrate, spinalised, unanaesthetised rats, as a measure of the development of chronic pain states. It was found that galanin may be useful in inhibiting the pain response. There is no mention of the use of GALR2 agonists to prevent or treat brain damage, injury or disease.

EP-A-0918455 discloses that recovery from crush injury (indicative of the regenerative abilities of sensory axons in the sciatic nerve), neuron survival during development and long term potentiation (LTP) are reduced in mice lacking the galanin gene compared to wild-type mice. From these results, it was proposed that galanin agonists may be suitable for use in the preparation of medicaments for the repair of nerve damage. There is also mention that a galanin agonist is useful in the treatment of Alzheimer's Disease and associated memory loss. No mention was made of which galanin receptor subtype mediates these effects, nor the effects of galanin agonists in protecting the central nervous system from injury, damage or diseases other than Alzheimer's Disease.

In addition, the above patent application, along with EP-A-1342410, describes a mammal, particularly a mouse, which has been engineered such that it lacks the galanin gene.

WO02/096934 discloses a series of galanin agonist compounds which may be used to treat convulsive seizures such as those which take place in epilepsy. There is mention that such compounds could be used for CNS injuries or in open heart surgery to prevent anoxic damage. However, there is no support for this, since all experimental results included in WO02/096934 relate to the treatment of convulsive seizures. The research group of which the inventors for that application were a part subsequently published information relating to one of these compounds, named “galnon” (Wu et al. (2003) Eur. J. Pharmacol. 482 133-137). Galnon equally activates and has agonistic activity to both GALR1 and GALR2. In addition, recent work shows that this compound also activates a number of other GPCR receptors including the neurotensin receptor (abstract Wang et al., Functional activity of galanin peptide analogues. Program No. 960.4 2004 Abstract Viewer/Itinerary Planner. Washington D.C.: Society for Neuroscience, 2004. Online. Thus galnon is not specific in its activation of galanin receptors nor is it a GALR2-specific agonist. The patent application WO02/096934 claims use of galnon in the treatment of pain, epilepsy, but makes no specific claim in relation to the use of such a compound in the treatment of brain injury, trauma or disease.

Saar et al. (2002) Proc. Natl. Acad. Sci. U.S.A. 99 7136-7141, Zachariou et al. (2003) Proc. Natl. Acad. Sci. U.S.A. 100 9028-9033 and Abramov et al. (2003) Neuropeptides 38 55-61 discuss the use of galnon in studies of epilepsy, opioid addiction and feeding, respectively.

### Galanin Receptors

Three G-protein coupled galanin receptor subtypes have been identified, GALR1, GALR2 and GALR3 (Habert-Ortoli et al. (1994) Proc. Natl. Acad. Sci. USA 91 9780-9783; Burgevin et al. (1995) J. Mol. Neurosci. 6 33-41; Howard et al. (1997) FEBS Letts. 405 285-290; Smith et al. (1997) J. Biol. Chem. 27-24616; Wang et al. (1997a) Mol. Pharmacol. 52 337-343; Wang et al. (1997b) J. Biol. Chem. 272 31949-31953; Ahmad et al. (1998) Ann. N.Y. Acad. Sci. 863 108-119; Bloomquist et al. (1998) Biophys. Res. Commun. 243 474-479; Kolakowski et al. (1998) J. Neurochem. 71 2239-2251; Smith et al. (1998) J. Biol. Chem. 273 23321-23326). Binding of galanin to GALR1 and GALR3 has been shown to inhibit adenylyl cyclase (Wang, 1998; Habert-Ortoli, 1994; Smith, 1998) by coupling to the inhibitory Gᵢ protein. In contrast, activation of GALR2 stimulates phospholipase C and protein kinase C activity by coupling to G_{q/11} (Fathi, 1997; Howard, 1997; Wang, 1997a; Wittau et al. (2000) Oncogene 19 4199-4209), hence activating the extracellular signal-regulated kinases (ERK) cascade. The negative coupling of GALR1 and GALR3 to adenylyl cyclase would be expected to have inhibitory effects on neuronal function after nerve injury or disease. In turn, this would be predicted to have negative and unwanted effects on behaviour and inhibit or delay recovery after injury and disease. Further, GALR1 and GALR3 are both expressed in the heart and gut, GALR1 also being expressed in the lung and bladder.

The lack of receptor subtype-specific antisera and the paucity of galanin ligands that are receptor subtype-specific, continues to hamper the analysis of the functional roles played by each receptor. A major advance in the field has been the discovery that galanin 2-11 peptide (termed AR-M1896) preferentially binds to GALR2 with a 500-fold specificity compared to GALR1 and with an almost complete loss of GALR1 activation (Liu et al. (2001) Proc. Natl. Acad. Sci. USA 98 9960-9964; Berger et al. (2004) Endocrinology 145 500-507). There is no published data as to whether AR-M1896 binds, or activates, GALR3. AR-M1896 has previously been used to demonstrate that activation of GALR2 appears to be the principal mechanism by which galanin stimulates neurite outgrowth from adult sensory neurons of the peripheral nervous system (Mahoney, 2003). Galanin 1-15 peptide and galanin 1-16 peptide are also known to be portions of the full-length galanin neuropeptide which will activate a galanin receptor.

Throughout this specification, the term “GALR” indicates a receptor which is one of the group of receptors consisting of GALR1, GALR2 and GALR3. The group includes, without limitation, the human, rat and mouse receptors. The receptor may also be chimaeric in form (i.e. including GALR sequences from different species), truncated (i.e. shorter than a native GALR sequence) or extended (i.e. including additional sequence beyond that of a native GALR sequence). Activation of the receptor may be determined, for example, by an increase in intracellular calcium levels.

Throughout this specification, the term “GALR2-specific agonist” indicates a substance capable of triggering a response in a cell as a result of the activation of GALR2 by the substance, but which does not activate (or activates with less potency) GALR1 and/or GALR3. Methods of identifying whether or not a compound is an agonist of a galanin receptor are known in the art, for example, Botella et al. (1995) Gastroenterology 108 3-11 and Barblivien et al. (1995) Neuroreport 6 1849-1852. A GALR2-specific agonist is one that preferentially binds and activates GALR2 with a selectivity of at least 30-fold compared to binding and activation of GALR1, preferably with greater than 50-fold selectivity over GALR1 and more preferably with greater than 100-fold selectivity over GALR1. The GALR2-specific agonist may also preferentially bind and activate GALR2 with a selectivity of at least 30-fold compared to binding and activation of GALR3, preferably with greater than 50-fold selectivity over GALR3 and more preferably with greater than 100-fold selectivity over GALR3.

### DISCLOSURE OF INVENTION

According to a first aspect of the invention, there is provided the use of a GALR2-specific agonist in the preparation of a medicament for the prevention or treatment of brain damage, injury or disease.

Advantageously, the use of a GALR2-specific agonist allows the prevention of brain damage, injury or disease, or an improvement in the condition of individuals who have suffered such brain damage, injury or disease, as a result of the ability of galanin and galanin agonists to reduce cell death in such situations. Galanin also acts as an endogenous neuroprotective factor to the hippocampus. A GALR2-specific agonist which does not activate GALR1 and/or GALR3 has benefits in treating brain injury or disease, minimizing unwanted or harmful peripheral side effects attributable to activation of GALR1 or GALR3, as the result of the different signaling cascades utilized by each of the three receptors.

The brain injury or damage may be caused by one of: embolic, thrombotic or haemorrhagic stroke; direct or indirect trauma or surgery to the brain or spinal cord; ischaemic or embolic damage to the brain during cardiopulmonary bypass surgery or renal dialysis; reperfusion brain damage following myocardial infarction; brain disease; immunological damage, chemical damage or radiation damage. The immunological damage may be the result of bacterial or viral infection. The chemical damage may be the result of excess alcohol consumption or administration of chemotherapy agents for cancer treatment. The radiation damage may be the result of radiotherapy for cancer treatment.

The brain disease is preferably one of Alzheimer's Disease, Parkinson's Disease, Multiple Sclerosis or variant Creutzfeld Jacob Disease.

The GALR2-specific agonist may be a polypeptide comprising a portion of the galanin amino acid sequence and preferably is AR-M1896.

Alternatively, the GALR2-specific agonist may be a non-peptide small chemical entity.

The GALR2-specific agonist may have a binding affinity for GALR2 of between 0 and 100 μM, preferably between 0 and 1 μM and has a greater than 30-fold binding specificity for GALR2 over GALR1, preferably greater than 50-fold binding specificity, most preferably greater than 100-fold binding specificity. The GALR2-specific agonist may also have greater than 30-fold binding specificity for GALR2 over GALR3, preferably greater than 50-fold binding specificity, most preferably greater than 100-fold binding specificity.

According to a second aspect of the invention, there is provided a method for preventing or treating brain injury, damage or disease comprising administering an effective amount of a GALR2-specific agonist to an individual in need of such prevention or treatment. Preferably, the individual is a human individual.

The brain injury or damage may be caused by one of: embolic, thrombotic or haemorrhagic stroke; direct or indirect trauma or surgery to the brain or spinal cord; ischaemic or embolic damage to the brain during cardiopulmonary bypass surgery or renal dialysis; reperfusion brain damage following myocardial infarction; brain disease; immunological damage, chemical damage or radiation damage. The immunological damage may be the result of bacterial or viral infection. The chemical damage may be the result of excess alcohol consumption or administration of chemotherapy agents for cancer treatment. The radiation damage may be the result of radiotherapy for cancer treatment.

The brain disease is preferably one of Alzheimer's Disease, Parkinson's Disease, Multiple Sclerosis or variant Creutzfeld Jacob Disease.

The GALR2-specific agonist may be a polypeptide comprising a portion of the galanin amino acid sequence and preferably is AR-M1896.

Alternatively, the GALR2-specific agonist may be a non-peptide small chemical entity.

The GALR2-specific agonist may have a binding affinity for GALR2 of between 0 and 100 μM, preferably between 0 and 1 μM and has a greater than 30-fold binding specificity for GALR2 over GALR1, preferably greater than 50-fold binding specificity, most preferably greater than 100-fold binding specificity. The GALR2-specific agonist may also have greater than 30-fold binding specificity for GALR2 over GALR3, preferably greater than 50-fold binding specificity, most preferably greater than 100-fold binding specificity.

According to a third aspect of the invention, there is provided a method of selecting a candidate brain injury, damage or repair prevention or treatment compound, comprising determining whether at least one test compound is a GALR2-specific agonist and selecting the at least one test compound as a candidate compound if it is a GALR2-specific agonist.

It may be determined that the at least one test compound binds to GALR2 with a binding affinity of between 0 and 100 μM, preferably between 0 and 1 μM The test compound is greater than 30-fold selective, preferably greater than 50-fold selective and most preferably greater than 100-fold selective for binding to GALR2 compared to binding to GALR1. Preferably, the test compound is also greater than 30-fold selective, preferably greater than 50-fold selective and most preferably greater than 100-fold selective for binding to GALR2 compared to binding to GALR3.

The GALR2 may comprise at least a portion of human GALR2, or may be full-length human GALR2.

The GALR2 may comprise at least a portion of non-human GALR2, preferably rat or mouse GALR2, or may be full-length GALR2.

The GALR2 may be a chimeric receptor construct.

Using a method according to this aspect of the invention, a selection of test compounds may be screened in a high throughput screening assay.

According to a fourth aspect of the invention, there is provided a pharmaceutical composition for use in the prevention or treatment of brain injury, damage or disease, the composition comprising:

a) an effective amount of at least one GALR2-specific agonist, or pharmaceutically acceptable salts thereof; and

b) a pharmaceutically suitable adjuvant, carrier or vehicle.

The brain injury or damage may be caused by one of: embolic, thrombotic or haemorrhagic stroke; direct or indirect trauma or surgery to the brain or spinal cord; ischaemic or embolic damage to the brain during cardiopulmonary bypass surgery or renal dialysis; reperfusion brain damage following myocardial infarction; brain disease; immunological damage, chemical damage or radiation damage. The immunological damage may be the result of bacterial or viral infection. The chemical damage may be the result of excess alcohol consumption or administration of chemotherapy agents for cancer treatment. The radiation damage may be the result of radiotherapy for cancer treatment.

The brain disease is preferably one of Alzheimer's Disease, Parkinson's Disease, Multiple Sclerosis or variant Creutzfeld Jacob Disease.

The GALR2 specific-agonist may be a polypeptide comprising a portion of the galanin amino acid sequence and preferably is AR-M1896.

Alternatively the GALR2 specific-agonist may be a non-peptide small chemical entity.

The GALR2 specific-agonist may have a binding affinity for GALR2 of between 0 and 100 μM, preferably between 0 and 1 μM and has a greater than 30-fold binding specificity for GALR2 over GALR1, preferably greater than 50-fold binding specificity, most preferably greater than 100-fold binding specificity. The GALR2-specific agonist may also have greater than 30-fold binding specificity for GALR2 over GALR3, preferably greater than 50-fold binding specificity, most preferably greater than 100-fold binding specificity.

The pharmaceutically suitable adjuvant, carrier or vehicle may be selected from: ion exchangers, alumina, aluminium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical composition may be administered orally or parenterally, preferably orally.

Where the pharmaceutical composition is administered orally, it may be in the form of a capsule or a tablet, and may preferably comprise lactose and/or corn starch. The pharmaceutical composition may further comprise a lubricating agent, preferably magnesium stearate. The pharmaceutical composition may be in the form of an aqueous suspension or aqueous solution, and may further comprise an emulsifying agent and/or a suspending agent. The pharmaceutical composition may comprise sweetening, flavouring and/or colouring agents.

The pharmaceutical composition may alternatively be administered by injection, by use of a needle-free device, by inhalation spray, topically, rectally, nasally, buccally, vaginally or via an implanted reservoir.

Where the pharmaceutical composition is administered by injection or needle-free device, it may be in the form of a sterile injectable preparation or a form suitable for administration by needle-free device. The sterile injectible preparation or form suitable for administration by needle-free device may be an aqueous or an oleaginous suspension, or a suspension in a non-toxic parenterally-acceptable diluent or solvent. The aqueous suspension may be prepared in mannitol, water, Ringer's solution or isotonic sodium chloride solution. The oleaginous suspension may be prepared in a synthetic monoglyceride, a synthetic diglyceride, a fatty acid or a natural pharmaceutically-acceptable oil. The fatty acid may be an oleic acid or an oleic acid glyceride derivative. The natural pharmaceutically-acceptable oil may be an olive oil, a castor oil, or a polyoxyethylated olive oil or castor oil. The oleaginous suspension may contain a long-chain alcohol diluent or dispersant, preferably Ph. Helv.

Where the pharmaceutical composition is administered rectally, it may be in the form of a suppository for rectal administration. The suppository may comprise a non-irritating excipient which is solid at room temperature and liquid at rectal temperature. The non-irritating excipient may be one of cocoa butter, beeswax or a polyethylene glycol.

Where the pharmaceutical composition is administered topically, it may be an ointment comprising a carrier selected from mineral oil, liquid petroleum, white petroleum, propylene glycol, polyoxyethylene-polyoxypropylene compounds, emulsifying wax and water. Alternatively, it may be a lotion or cream comprising a carrier selected from mineral oil, sorbitan monostearate, polysorbate 60, cetyl esters wax, cetearyl alcohol, 2-octyldodecanol, benzyl alcohol and water.

Where the pharmaceutical composition is administered nasally, it may be administered by nasal aerosol and/or inhalation.

According to a fifth aspect of the invention, there is provided a method of inhibiting the death of a cell comprising contacting the cell with an amount of a GALR2-specific agonist effective to inhibit the death of the cell. The cell may a neuron, preferably a neuron from the central nervous system, preferably a hippocampal or cortical neuron. Preferably, the cell is a human cell. In this method, the death of a cell is inhibited as the result of the activation of GALR2 present in the cell. The death of a cell is inhibited if the probability of the occurrence of the cell's death is decreased and/or if the life of the cell is prolonged.

### BRIEF DESCRIPTION OF DRAWINGS

Embodiments of the invention will now be described, by way of example only, with reference to the accompanying FIGS. 1-4, in which:

FIG. 1 shows the effects of intraperitoneal administration of 20 mg/Kg kainate on hippocampal cell death in vivo;

FIG. 2 (A-C) shows the responses of galanin knockout, over-expressing and wild-type hippocampal cultures in vitro after incubation with 10 nM-1 μM staurosporine (St);

FIG. 3 (A-C) shows the effect of co-administration of staurosporine or glutamate with galanin or AR-M1896 on galanin wild-type hippocampal cultures in vitro; and

FIG. 4 (A-B) shows the responses of galanin knockout, over-expressing and wild-type animals in the Experimental Autoimmune Encephalomyelitis (EAE) model of MS in vivo.

### MODES OF CARRYING OUT THE INVENTION

### Methods

### Animals

All animals were fed standard chow and water ad libitum. Animal care and procedures were performed within the United Kingdom Home Office protocols and guidelines.

### Galanin Knockout Mice

Details of the strain and breeding history have been published previously (Wynick et al. (1998) Proc. Natl. Acad. Sci. USA 95 12671-12676). In brief, mice homozygous for a targeted mutation in the galanin gene were generated using the E14 cell line. A PGK-Neo cassette in reverse orientation was used to replace exons 1-5, and the mutation was bred to homozygosity and has remained inbred on the 129OlaHsd strain. Age and sex matched wild-type littermates were used as controls in all experiments.

### Galanin Over-Expressing Mice

Details of the strain and breeding history have been published previously (Bacon et al. (2002) Neuroreport 13 2129-2132) In brief, galanin over-expressing mice were generated on the CBA/B6 F1 hybrid background. A mouse 129 sv cosmid genomic library was screened and a ˜25 kb region was subcloned which contained the entire murine galanin coding region and ˜20 kb of upstream sequence. The transgene was excised by restriction digest and microinjected into fertilised oocytes at 5 ng/μl final concentration. Four galanin over-expressing transgenic lines were generated as previously described (Bacon et al. (2002) Neuroreport 13 2129-2132) and galanin expression in the hippocampus was assessed by immunocytochemistry (see below). Line 46 was found to have highest levels of galanin expression in the CA1 and CA3 regions of the hippocampus and in the dentate gyrus compared to the three other lines and wild-type controls. Line 46 was therefore used for all subsequent experiments.

### Organotypic Hippocampal Cultures

Organotypic cultures were prepared as previously described (Elliott-Hunt et al. (2002) J. Neurochem. 80 416-425; Stoppini et al. (1991) J. Neurosci. Methods 37 173-182). Briefly, the hippocampi from 5-6 day old pups were rapidly removed under a dissection microscope and sectioned transversely at 400 μm using a McIlwain tissue chopper (Mickle Laboratory Engineering Co. Ltd., Gomshall, UK). The slices were cultured in 95% air and 5% CO₂ at 37° C. on a microporous transmembrane biopore membrane (Millipore, Poole, UK), in a 6-well plate, in 50% minimal essential medium with Earle's Salts (Gibco BRL, Paisley, UK) without L-glutamine, 50% Hanks Balanced Salt Solution (Gibco BRL), 25% Horse Serum (heat inactivated; Harlan Serum Labs, Loughborough, UK), 5 mg/ml glucose (Sigma Chemical Co., Poole, UK) and 1 ml glutamine (Sigma).

### Preparation of Primary Neuronal Cultures

Hippocampi from 2-3 day old pups were dissected and placed into 4° C. collection buffer prepared with Hanks Balanced Salt Solution (calcium and magnesium free) (Gibco BRL, Paisley, UK), 10% (v/v) N-2-Hydroxyethylpiperazine-N′-2-ethanesulfonic acid (HEPES) (ICN Biomedicals Inc., Aurora, Ohio, USA), 50 U/ml penicillin (Britannia Pharmaceuticals Ltd., Redhill, Surrey, UK), 0.05 mg/ml streptomycin in 100 ml (Sigma Chemical Company, Poole, Dorset, UK), and 0.5% (v/v) Bovine Serum Albumin (BSA; ICN Biomedicals Inc., Aurora, Ohio, USA). Enzymatic digestion, isolation and culture of hippocampal neurons was performed as previously described (McManus & Brewer (1997) Neurosci. Lett. 224 193-196). Cells were counted and plated at 40,000 cells/well onto D-L-poly-ornithine (Sigma) coated 96 well plates. After 24 hours 10 μg/ml 5′Fluoro 2′ Deoxyuridine (Sigma; anti-mitotic agent) was added. Cultures were incubated at 37° C. with ambient oxygen and 5% CO₂ for 9 days before experimentation. The media was changed after the first 3 days and then every fourth day thereafter.

### Immunohistochemistry

Mice were intracardially perfused with 4% paraformaldehyde/Phosphate Buffered Saline (PBS). The brains were removed and post-fixed for 4 hours at room temperature. The brains were then equilibrated in 20% sucrose overnight at 4° C., embedded in Optimal Cutting Temperature (OCT) compound (Tissue Tek Ltd., Eastbourne, UK) mounting medium, frozen on dry ice, and cryostat-sectioned (30 μm sections). Sections were blocked and permeabilised in 10% normal goat serum/PBS 0.2% Triton X-100 (PBST) for 1 hour at room temperature. Sections were then incubated in rabbit polyclonal antibody to galanin (Affinity, Nottingham, UK) at 1:1000 in PBST overnight at room temperature, washed 3×10 minutes in PBS, and incubated in fluorescein isothiocyanate (FITC)-goat (The Jackson Laboratory, Westgrove, Pa., USA) at 1:800 for 3 hours at room temperature. After washing, sections were mounted in Vectashield™ (Vector Laboratories Inc., Burlington, Calif., USA). Images were taken by using a Leica fluorescent microscope (Leica Microsystems, Milton Keynes, UK) with RT Color Spot camera and Spot Advance image capture system software (Diagnostic Instruments, Sterling Heights, Mich., USA).

Galanin immunohistochemistry was also performed on dispersed hippocampal neurons and organotypic cultures which were fixed in 4% paraformaldehyde, permeabilised with Triton X-100 and then processed as above.

### Staurosporine And Glutamate Induced Hippocampal Damage

Fourteen day organotypic hippocampal cultures were placed in 0.1% BSA with serum free media for 16 hours before incubation with varying concentrations of glutamic acid for 3 hours or staurosporine for 9 hours. Staurosporine and glutamate are both known to cause excitotoxic damage to such cell cultures (Prehn et al. (1997) J. Neurochem. 68 1679-1685; Ohmori et al. (1996) Brain Res. 743 109-115). Cultures were washed with serum-free medium and incubated for a further 24 hours before imaging. Regional patterns of neuronal injury in the organotypic cultures were observed by performing experiments in the presence of propidium iodide. After membrane injury, the dye enters cells, binds to nucleic acids and accumulates, rendering the cell brightly fluorescent (Vornov et al. (1994) Stroke 25 457-465). The CA1 neuronal subfield was clearly visible in a bright field image. Neuronal damage in the area encompassing the CA1 region was assessed using the density slice function in NIH Image software (Scion Image, MD, USA) to establish signal above background. The area of the subfields expressing the exclusion dye propidium iodide was measured, and expressed as a percentage of the total area of the subfields as assessed in the bright field image. Furthermore, for consistency in setting the parameters accurately when using the density slice function, the threshold was set against a positive control set of cultures exposed to 10 mM glutamate.

Nine-day primary hippocampal cultures were exposed to staurosporine for 24 hours. The viability of neurons was measured by manual counting of both live and dead neurons using a live/dead kit (Molecular Probes, Lieden, Netherlands).

### Treatments

Organotypic or dispersed primary hippocampal cultures were at various times cultured with or without the addition of the following chemicals: staurosporine (Sigma), L-glutamic acid (Sigma), galanin peptide (Bachem, Merseyside, UK), the high-affinity GALR2-specific agonist AR-M1896 [Gal(2-11)Trp-Thr-Leu-Asn-Ser-Ala-Gly-Tyr-Leu-Leu-NH2] (SEQ ID NO: 1) (AstraZeneca, Montreal, Quebec, Canada), amyloid-β (1-42) (Aβ (1-42)) and the reverse Aβ (42-1) peptide (American Peptide Company, Sunnyvale, Calif. 93906). Before use in the experiments below, the Aβ (1-42) was induced to form fibrils by pre-incubation in culture medium. Specifically, 0.45 mg of Aβ peptide was dissolved in 20 μl of dimethyl sulfoxide (DMSO-Sigma) and diluted to a 100-μM stock solution in medium, which was then incubated with gentle shaking at room temperature for 24 hours.

### Kainate-Induced Hippocampal Injury

8-week old female mice were injected with intraperitoneal (i.p.) kainic acid (Tocris Cookson, Bristol, UK) (20 mg/kg) or vehicle (PBS, 1 ml/kg). Kainic acid is known to cause hippocampal damage as previously described (Beer et al. (1998) Brain Res. 794 255-266; Mazarati et al (2000) J. Neurosci. 16 6276-6281). Hippocampal cell death was measured by terminal deoxynucleotidyl transferase-mediated fluorescein-dUTP nick end labelling (TUNEL). Animals were killed at 72 hours after injection with kainic acid or vehicle. Mice were intracardially perfused with 4% paraformaldehyde/PBS and the brains rapidly removed and post fixed for 4 hours at room temperature. The brains were equilibrated in 20% sucrose overnight at 4° C., embedded in OCT mounting media and frozen on dry ice. Sections were cut (16 μm) on a cryostat, thaw mounted onto gelatine coated slides and stored at −80° C. until use. Apoptosis was evaluated by using an in situ cell detection kit (Boehringer, Berkshire, UK). Every sixth section was collected and blocked with methanol and permeabilised with triton (0.1%) and sodium citrate (0.1%) and then labelled with fluorescein dUTP in a humid box for 1 hour at 37° C. The sections were then combined with horse radish peroxidase, colocalised with diaminobenzidine (DAB) and counterstained with haemytoxin. Controls received the same management except the labelling omission of fluorescein dUTP. After washing, sections were mounted in Vectashield™ (Vector Labs Inc.). Cells were visualised using a Leica fluorescent microscope with RT Colour Spot camera and Spot Advance image capture system software (Diagnostic Instruments Inc., Sterling Heights, Mich., USA).

### EAE Model

The standard EAE model of MS was used as previously described (Radu et al. (2000) Int. Immunol. 12 1553-60). Mice were immunized subcutaneously in one hind leg with a total of 200 μg of MBP 1-9 (AcASQKRPSQR, (SEQ ID NO: 2) synthesized by Abimed, Langenfeld, Germany), emulsified with complete Freund's adjuvant (Sigma) supplemented with 4 mg/ml Mycobacterium tuberculosis strain H37RA (Difco, Detroit, Mich.). M. tuberculosis purified protein derivative (PPD) was obtained from the UK Central Veterinary Laboratory (Weybridge, UK). Mice were scored for symptoms of EAE as follows: 0, no signs; 1, flaccid tail; 2, partial hind limb paralysis and/or impaired righting reflex; 3, full hind limb paralysis; 4, hind limb plus fore limb paralysis; and 5, moribund or dead.

### Statistical Analysis

Data are presented as the mean±SEM. Student's t test was used to analyse the difference in staurosporine concentrations within groups. ANOVAs or non-parametric Mann-Whitney U post hoc tests were used as appropriate to analyse differences between genotypes and different ligands and/or staurosporine and glutamate points. A P value of <0.05 was considered to be significant.

### Candidate Compound Screening Method

CHO cells transfected with and stably expressing the cDNA encoding either the human GALR1, GALR2 or GALR3 were obtained from Euroscreen (Brussels, Belgium). Cells were cultured in Nutrient Mix (HAMS) F12 (Gibco BRL, Paisley, UK), supplemented with 10% foetal bovine serum (Gibco BRL) and 0.4 mg/ml G418 (Sigma) in 3 layer culture flasks at 37° C. in a 5% CO₂/95% air atmosphere. Cells were grown to approximately 80% confluence and dissociated in 0.02% EDTA in D-PBS for 10 minutes at 37° C. Cells were collected by centrifugation at 1000 rpm for 5 minutes and then resuspended in medium to the required density on the day of the experiment. Cellular responses to the addition of various compounds were then measured using a FLIPR384 (Molecular Devices Ltd, Wokingham, UK). Cells were suspended in culture medium at a density of 20,000 cells/30A transferred to 384 well black/clear Greiner culture plates (30 μl/well) and incubated at 37° C. in a 5% CO₂/95% air humidified atmosphere for 2 hours. Cells were loaded with dye by the addition of 30 μl Fluo-4-AM (4 μM in assay buffer with 0.8% pluronic F-127 and 1% FBS) to each well and incubated at 37° C. in a 5% CO₂/95% air humidified atmosphere for 1 hour. Cells were washed in FLIPR assay buffer (HBSS without calcium or magnesium with the addition of 20 mM Hepes, 1 mM MgCl₂, 2 mM CaCl₂, 2.5 mM Probenecid and 0.1% BSA) using an EMBLA plate washer (4×80 μl washes) such that 45 μl remained in each well after washing.

Responses to compounds were measured using a FLIPR384. Basal fluorescence was recorded for every second for 10 seconds prior to compound addition (5 μl; final concentration 10 μM) and fluorescence recorded every second for 60 readings then every 6 seconds for a further 20 readings. Data were recorded as relative fluorescence units (RFU) and analysis was performed on exported statistics recording maximum RFU over the 3 min recording. Data were analysed using XLFit 3.0. All data were subjected to the relevant quality control (QC) procedure prior to release. EC50 for each compound was calculated for each of the GALR expressing cell lines and from that data, compounds which acted as GALR2-specific-agonists were identified.

### Results

### Experiment 1

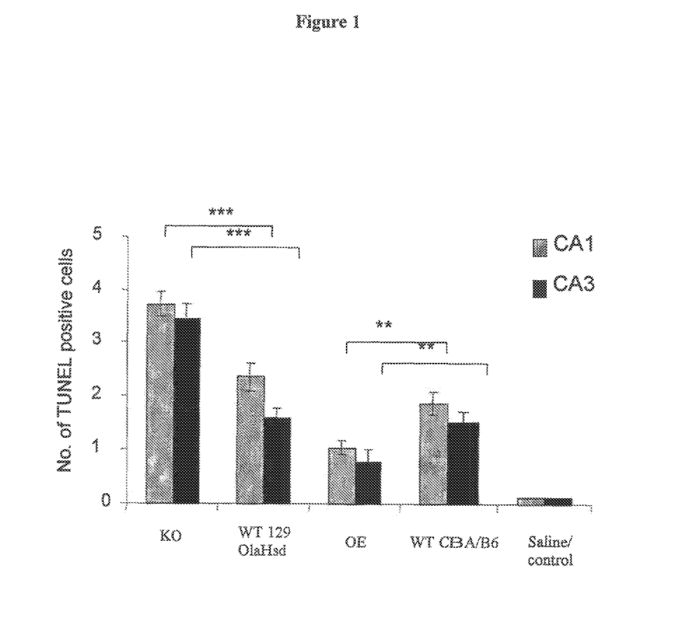

Intraperitoneal administration of 20 mg/kg kainic acid was used to induce excitotoxic hippocampal damage as previously described (Beer, 1998; Mazarati, 2000; Tooyama et al. (2002) Epilepsia 43 Suppl 9 39-43). Three days later brains were harvested and hippocampal cell death assessed by counting the number of TUNEL-positive cells. The results are displayed in FIG. 1. The number of apoptotic neurons was significantly greater in both the CA 1 and CA3 regions of the galanin knockout animals (KO) compared to the strain-matched wild-type controls (WT) (FIG. 1), an increase of 62.9% and 44.8% respectively (**P<0.01, ***p<0.001). Conversely, the degree of cell death was significantly lower in both the CA1 and CA3 regions of the galanin over-expressing animals (OE) than in strain matched controls (WT) (FIG. 1), a decrease of 55.6% and 50.4% respectively (p<0.05).

### Experiment 2

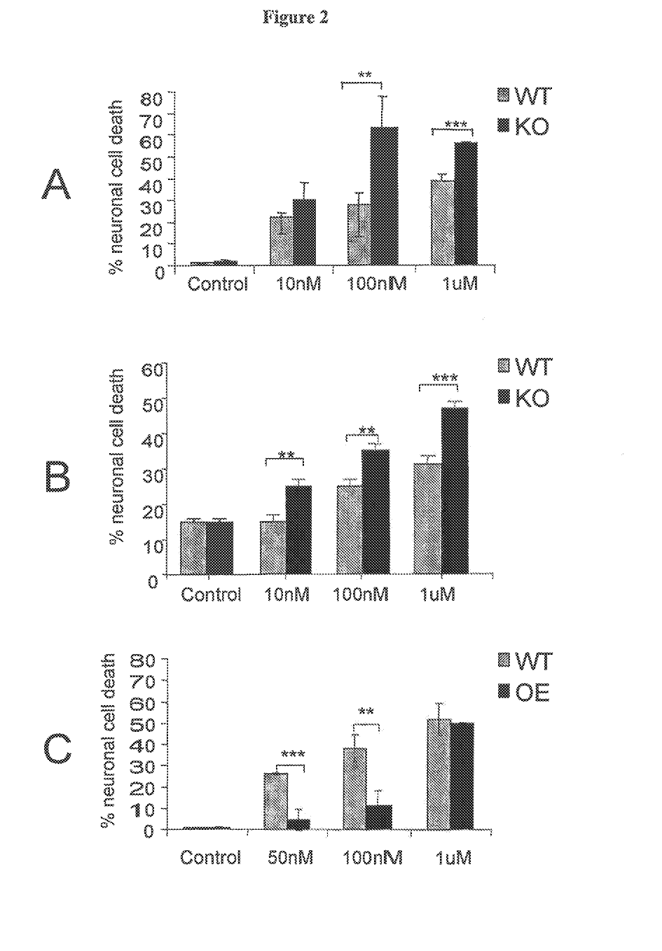

To further dissect the neuroprotective role played by galanin in a more tractable in vitro system, both primary dispersed and organotypic hippocampal cultures (Elliott-Hunt, 2002) were used. These two techniques are complimentary since the dispersed hippocampal cultures ensure that observed effects are neuron-specific, whilst the organotypic cultures preserve the synaptic and anatomical organisation of the neuronal circuitry (Elliott-Hunt, 2002) as well as retaining many of the functional characteristics found in vivo (Adamschik et al. (2000) Brain Res. Prot. 5 153-158). The effects of staurosporine and glutamate on neuronal cell death in hippocampal cultures (Prehn, 1997; Ohmori, 1996) were studied. Cell death was visualised by propidium iodide staining. Results are expressed as a percentage of the area expressing fluorescence as compared with the untreated “control” cultures. Staurosporine at 1 μM and 100 nM caused significant and consistent levels of neurotoxicity in both the wild-type (WT) and galanin knockout (KO) cultures. The percentage cell death was significantly higher in galanin knockout animals compared to wild-type controls at both doses (1 μM: 68±0.5% vs 38±8%; 100 nM: 65±10% vs 40±26%; n=4, p<0.05), as shown in FIG. 2A. Similarly, a marked and significant excess of cell death in the galanin knockout organotypic cultures after 9 hour exposure to 4 mM glutamate was noted, compared to wild-type controls (85±8.6% vs 61±9.3%; n=4, p<0.05).

To ensure that the above effects were neuron-specific, the effects of staurosporine in dispersed primary hippocampal neurons were also studied. Once again a significant excess of cell death in the galanin knockout cultures was observed, compared to wild-type controls (n=4, p<0.01), over the range of 10 nM-1 μM staurosporine (FIG. 2B).

### Experiment 3

Having demonstrated that an absence of galanin increases the susceptibility to hippocampal cell death, the studies were extended to the galanin over-expressing mice. A significant reduction in cell death was observed in the galanin over-expressing animals (OE) after exposure to 50 nM or 100 nM staurosporine, compared to strain-matched wild-type controls (WT) (FIG. 2C; n=4, **p<0.01, ***p<0.001)

### Experiment 4

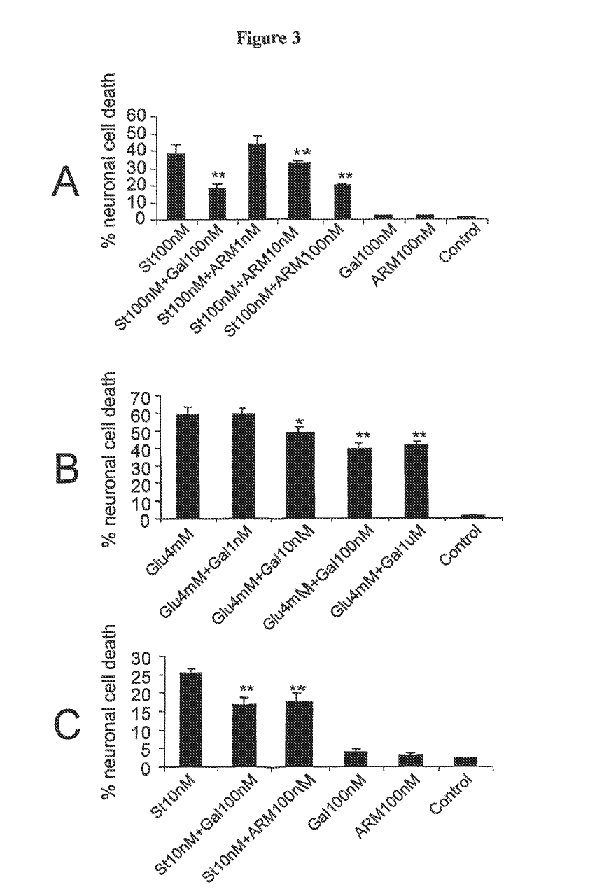

To test whether exogenous galanin would protect wild-type hippocampal neurons from damage, 100 mM galanin was co-administered with 100 nM staurosporine to wild-type organotypic cultures. This co-administration provided significant neuroprotection (n=4, p<0.05) in these cultures (FIG. 3A). Similarly, galanin was also protective over the dose range 10 nM-1 μM when co-administered with 4 mM glutamate in wild-type organotypic cultures (FIG. 3B). In keeping with these findings using organotypic cultures, 100 nM galanin also protected wild-type dispersed primary hippocampal neurons from cell death induced by 10 nM staurosporine (FIG. 3C; n=3, p<0.05)

### Experiment 5

The neuroprotective effects of galanin in the hippocampus are likely to be mediated by activation of one or more of three G-protein coupled galanin receptor subtypes, GALR1, GALR2 and GALR3. It has previously been shown that activation of GALR2 appears to be the principal mechanism by which galanin stimulates neurite outgrowth from adult sensory neurons (Mahoney, 2003). Therefore, the effect of 100 nM AR-M1896 (a high-affinity GALR2-specific agonist), when co-administered with 100 nM staurosporine in organotypic cultures from wildtype animals, was also tested. It should be noted that even if AR-M1896 does weakly activate GALR1, this would be most unlikely at 100 nM when the IC₅₀ for GALR1 is 879 nM. AR-M1896 significantly reduced the amount of cell death in wild-type organotypic cultures to a similar amount observed with equimolar concentrations of galanin (p<0.05, FIG. 3A). The addition of AR-M1896 was also as effective in reducing staurosporine-induced cell death in galanin knockout cultures as that observed in the wild-type organotypic cultures (data not shown). Dispersed primary hippocampal neurons were also treated with AR-M1896 and staurosporine, demonstrating similar protective effects of the peptide to that observed with full-length galanin (FIG. 3C). No significant effects of galanin or AR-M1896 were noted in the absence of staurosporine in organotypic or primary cultures

### Experiment 6

Disease progression in AD is associated with the deposition of amyloid-β fibrils in the brain to form senile plaques consisting of peptides derived from the cleavage of the amyloid precursor protein by a-secretases (Gamblin et al. (2003) Proc. Natl. Acad. Sci. U.S.A. 100 10032-10037). Deposits of fibrillar amyloid-β are assumed to have a causative role in the neuropathogenesis of AD. To test whether endogenous galanin lays a protective effects on neuronal toxicity induced by fibrillar Aβ, 14 day old hippocampal organotypic cultures were obtained from galanin knockout, galanin over-expressing and strain matched wild-type controls transgenic animals. These cultures were treated for up to 72 hours with 10 μM fibrillar Aβ (1-42), the reverse control peptide Aβ (42-1) or the addition of no peptide. 10 μM fibrillar Aβ (1-42) was used as previously described (Zheng et al. (2002) Neuroscience 115 201-211). Experiments were performed in triplicate and cell death was measured as above using propidium iodide fluorescence (PIF) intensity. Images were captured and analysed using Scion Image analysis software. The results demonstrate a statistically greater amount of fibrillar Aβ (1-42)-induced hippocampal cell death in the galanin knock-out animals compared to wild-type controls. Conversely, significantly less fibrillar Aβ (1-42)-induced hippocampal cell death was noted in the galanin over-expressing animals compared to strain-matched wild-type controls.

### Experiment 7

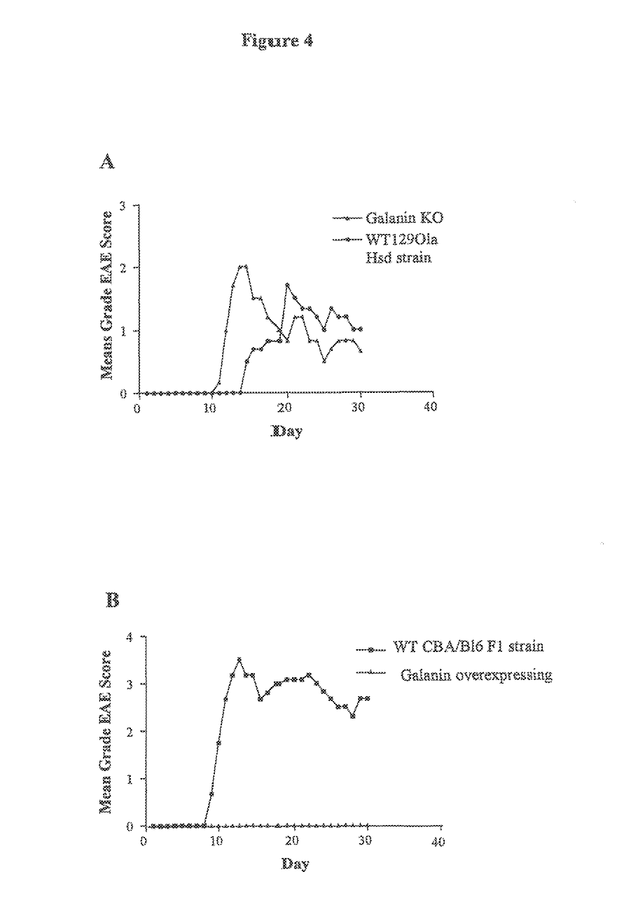

MS phenotype was induced in galanin knock-out, galanin over-expressing and strain matched wild-type control transgenic animals, using the previously described EAE model described above. FIG. 4A demonstrates that the galanin knockout animals develop an accelerated and more severe form of the disease compared to strain matched wildtype controls (N=5, P<0.01). Conversely, the galanin over-expressing mice fail to develop any symptoms of the disease in marked contrast to their wildtype controls (FIG. 4B; N=5, P<0.001). These data demonstrate once again that galanin plays a protective role in an inflammatory model of neuronal injury in the central nervous system.

### SUMMARY

It has been demonstrated that galanin acts as an endogenous neuroprotective factor to the hippocampus, in a number of in vivo and in vitro models of injury. Further, exogenous galanin and a previously described high-affinity GALR2-specific agonist both reduced cell death. Therefore, GALR2 is the principal receptor subtype that mediates these protective effects. These data indicate that a GALR2-specific agonist will have therapeutic uses in the treatment or prevention of various forms of brain injury, damage or disease.

1-16. (canceled)

17. A method for treating brain injury, damage or disease comprising administering an effective amount of a GALR2-specific agonist to an individual in need of such treatment.

18-32. (canceled)

33. A method of selecting a candidate brain injury, damage or repair treatment compound, comprising determining whether at least one test compound is a GALR2-specific agonist and selecting the at least one test compound as a candidate compound if it is a GALR2-specific agonist.

34. The method of claim 33, wherein it is determined that the at least one test compound binds to GALR2 with a binding affinity of between 0 and 100 μM and with a specificity of greater than 30-fold for GALR2 over GALR1.

35. The method of claim 33, wherein it is determined that at least one test compound binds to GALR2 with a binding affinity between 0 and 100 μM and with a specificity of greater that 50 fold for GALR2 over GALR1.

36. The method of claim 33, wherein it is determined that at least one test compound binds to GALR2 with a binding affinity between 0 and 100 μM and with a specificity of greater that 100 fold for GALR2 over GALR1.

37. The method of claim 34, wherein it is determined that at least one test compound binds to GALR2 with a specificity of greater than 30 fold for GALR2 over GALR3.

38. The method of claim 34, wherein it is determined that at least one test compound binds to GALR2 with a specificity of greater than 50 fold for GALR2 over GALR3.

39. The method of claim 34, wherein it is determined that at least one test compound binds to GALR2 with a specificity of greater than 100 fold for GALR2 over GALR3.

40. The method of claim 34, wherein it is determined that the at least one test compound binds to GALR2 with a binding affinity of between 0 and 1 μM.

41. The method of claim 33, wherein the GALR2 comprises at least a portion of human GALR2.

42. The method of claim 41, wherein the GALR2 is full-length human GALR2.

43. The method of claim 33, wherein the GALR2 comprises at least a portion of non-human GALR2.

44. The method of claim 43, wherein the GALR2 is rat or mouse GALR2.

45. The method of claim 43, wherein the GALR2 is full-length GALR2.

46. The method of claim 33, wherein the GALR2 is a chimeric receptor construct.

47. The method of claim 33, wherein a selection of test compounds are screened in a high throughput screening assay.

48. A pharmaceutical composition comprising:
a) an effective amount of at least one GALR2-specific agonist, or pharmaceutically acceptable salts thereof; and
b) a pharmaceutically suitable adjuvant, carrier or vehicle.

49-95. (canceled)

96. A method of inhibiting the death of a cell comprising contacting the cell with an amount of a GALR2-specific agonist effective to inhibit the death of the cell.

97-100. (canceled)
